SEQUENCE LISTING (2 sequences)

SEQ ID NO: 1 - DNA, 1700 bp, Homo sapiens
  modified_base (527)..(529): a, c, g, t, unknown or other
cttgcctgcaaacctttacttctgaaatgacttccacggctgggacgggaaccttccacccacagctatgcctctgattggtgaatggtgaaggtgcctgtctaacttttctgtaaaaagaaccagctgcctccaggcagccagccctcaagcatcacttacaggaccagagggacaagacatgactgtgatgaggagctgctttcgccaatttaacaccaagaagaattgaggctgcttgggaggaaggccaggaggaacacgagactgagagatgaattttcaacagaggctgcaaagcctgtggactttagccagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggatnnnatcacgagtgcccggctgctgcagcaggaggttctgcagnnngtctcggatgctgagagctgttaccttgtccacaccctgctggagttctacttgaaaactgttttcaaaaactaccacnnnagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctgaatgtctagaccaggacctccctccccctggcactggtttgttccctgtgtcatttcaaacagtctcccttcctatgctgttcactggacacttcacgcccttggccatgggtcccattcttggcccaggattattgtcaaagaagtcattctttaagcagcgccagtgacagtcagggaaggtgcctctggatgctgtgaagagtctacagagaagattcttgtatttattacaactctatttaattaatgtcagtatttcaactgaagttctatttatttgtgagactgtaagttacatgaaggcagcagaatattgtgccccatgcttctttacccctcacaatccttgccacagtgtggggcagtggatgggtgcttagtaagtacttaataaactgtggtgctttttttggcctgtctttggattgttaaaaaacagagagggatgcttggatgtaaaactgaacttcagagcatgaaaatcacactgtctgctgatatctgcagggacagagcattggggtgggggtaaggtgcatctgtttgaaaagtaaacgataaaatgtggattaaagtgcccagcacaaagcagatcctcaataaacatttcatttcccacccacactcgccagctcaccccatcatccctttcccttggtgccctccttttttttttatcctagtcattcttccctaatcttccacttgagtgtcaagctgaccttgctgatggtgacattgcacctggatgtactatccaatctgtgatgacattccctgctaataaaagacaacataactca

SEQ ID NO: 2 - protein, 206 aa, Homo sapiens
  MOD_RES (85): variable amino acid
MNFQQRLQSLWTLARPFCPPLLATASQMQMVVLPCLGFTLLLWSQVSGAQGQEFHFGPCQVKGVVPQKLWEAFWAVKDTMQAQDXITSARLLQQEVLQXVSDAESCYLVHTLLEFYLKTVFKNYHXRTVEVRTLKSFSTLANNFVLIVSQLQPSQENEMFSIRDSAHRRFLLFRRAFKQLDVEAALTKALGEVDILLTWMQKFYKL

(19) United States
(12) Patent Application Publication
(10) Pub. No.: US 2010/0120153 A1
(43) Pub. Date: May 13, 2010

(54) N-GLYCOSYLATION MUTANT OF MELANOMA DIFFERENTIATION ASSOCIATED GENE-7

(76) Inventors: Rahul V. Gopalkrishnan, Singapore (SG); Moira Sauane, New York, NY (US); Paul B. Fisher, Richmond, VA (US)

Correspondence Address: BAKER BOTTS L.L.P., 30 ROCKEFELLER PLAZA, 44TH FLOOR, NEW YORK, NY, 10112-4498, US

(21) Appl. No.: 12/434,387

(22) Filed: May 1, 2009

Related U.S. Application Data
(63) Continuation of application No. PCT/US07/83491, filed on Nov. 2, 2007.
(60) Provisional application No. 60/856,706, filed on Nov. 3, 2006.

(51) Int. Cl. C12N 15/00; C07H 21/00; C07K 14/00; C12N 15/63; C12N 5/00

(52) U.S. Cl. 435/455; 536/22.1; 530/350; 435/320.1; 435/375

(57) ABSTRACT

The present invention relates to MDA-7 variant proteins which are deficient in or lack glycosylation. It is based, at least in part, on the results of experiments which have demonstrated that such variants are functionally equivalent to wild-type MDA-7 protein. Such proteins may be more easily produced large-scale than the wild-type protein, and may be found to be less immunogenic (thereby facilitating treatment, especially repeated treatments). Accordingly, the present invention provides for such proteins (‘Gly(def)MDA-7 proteins’), nucleic acids encoding them (‘Gly(def)mda-7 nucleic acids), pharmaceutical compositions comprising such proteins or nucleic acids, and related methods of treatment.

### CROSS-REFERENCE TO RELATED APPLICATIONS

This application is a continuation of International Application PCT/US07/083491, filed Nov. 2, 2007, which claims priority to U.S. Provisional Application Ser. No. 60/856,706, filed Nov. 3, 2006, the contents of each of which are incorporated herein.

### SEQUENCE LISTING

The specification further incorporates by reference the Sequence Listing submitted herewith via EFS on May 1, 2009. Pursuant to 37 C.F.R. §1.52(e)(5), the Sequence Listing text file, identified as “0700503786.TXT,” is 5,064 bytes and was created on May 1, 2009.

### GRANT INFORMATION

The subject matter described herein was developed, at least in part, with the support of NIH/NCI Grants R01 CA097318, R01 CA098172, and P01 CA104177, so that the United States Government holds certain rights herein.

### 1. TECHNICAL FIELD

The present invention relates to variants of MDA-7/IL-24 protein which are deficient in or which lack glycosylation.

### 2. BACKGROUND OF THE INVENTION

A novel tumor cell-specific apoptosis-inducing gene, melanoma differentiation associated gene-7 (mda-7), was identified by subtraction hybridization from human melanoma cells induced to growth arrest and terminally differentiate by treatment with fibroblast interferon and mezerein (1, 2). Initial studies confirmed that expression of this gene correlated with induction of irreversible growth arrest, cancer reversion and terminal differentiation in human melanoma cells (1, 2). Additional investigations have confirmed its gene therapeutic potential in other human cancers, including malignant glioma and breast, prostate and ovary cancer-derived cells (3-7). Several independent studies demonstrated that a majority of human cancer-derived cell lines, including prostate, breast, cervical, lung, fibrosarcoma, colorectal, melanoma, and glioblastoma, undergo apoptosis when exposed to mda-7/IL-24 (3-7). In contrast, no significant growth inhibitory effect occurred when this gene was transduced into normal human breast or prostate epithelial, endothelial, melanocyte, astrocyte or fibroblast cells (3-7). This property of mda-7/IL-24 suggested translational potential for the gene-based therapy of multiple cancers. Moreover, based on pre-clinical cell culture and animal modeling studies, successful Phase I trials have now been performed and a Phase II clinical trial is in preparation (6-9). MDA-7/IL-24 has been delivered to cells, tumor xenografts and patient tumors in clinical trials via a nonreplicating adenovirus (Ad.mda-7). These studies are contributing significantly to our understanding of the underlying basis of mda-7/IL-24 activity. Elucidation of the mechanistic basis of the selective anti-tumor action of this novel cytokine will provide valuable insights ensuring safe use, improving efficacy, identifying potential pharmacological adjuvants or substitutes including small molecule mimetics and possibly uncovering important additional information for developing enhanced therapeutic applications (4).

Structural and motif sequence homology, in addition to functional conservation and chromosomal localization, indicated that mda-7 belongs to the IL-10 gene family of cytokines and has therefore been designated IL-24 (2, 4-7, 10-14). We previously demonstrated that mda-7/IL-24's cancer cell-specific activity could occur through mechanisms independent of binding to its currently recognized cognate receptors and might even occur independent of receptor function (15). Follow-up studies assessed whether the potent pro-apoptotic activity observed with Ad.mda-7 was due to intracellular or secreted MDA-7/IL-24 protein (16). We confirmed that Ad.mda-7 infection of cancer cells promoted ER-stress and demonstrated that mda-7/IL-24-mediated apoptosis could be triggered through an intracellular mechanism (confirmed by deletion of the signal peptide'of the mda-7/IL-24 sequence) and occurred efficiently in the absence of protein secretion (16). A potential intracellular mode of killing was further confirmed using a bacterially expressed and purified GST-MDA-7 fusion protein (17).

Most secreted proteins of eukaryotic cells enter the secretory pathway through the translocation channel at the membrane of the endoplasmic reticulum (ER) (18). The lumen of the ER is the site where translocated proteins assume their secondary structure and where assembly of oligomeric complexes occurs. It is there where cotranslational and posttranslational modifications also occur. Once proteins acquire their fully folded native conformation they can proceed in the secretory pathway. Many of the proteins that fold in the ER are covalently modified by the co-translational addition of N-linked glycans that contribute not only to their conformational maturation but also to their multiple biological functions (19, 20). In fact, glycans provide polar surface groups, thus enhancing the solubility and preventing the aggregation of the polypeptide, on one hand, and enabling the nascent glycoproteins to interact with a number of ER-resident chaperones, on the other (18, 19).

The mRNA encoding mda-7/IL-24 is ˜2-kb in length, generating a predicted protein of 23.8-kD belonging to the four-helix bundle family of cytokine molecules (2, 12). The open reading frame encodes a molecule that is 206-amino acids in length, which is a precursor form of the ultimate cleaved, post-translationally processed and secreted mature product. There are three consensus asparagine glycosylation residues at amino acid 85, 99 and 126, which are N-glycosylated resulting in a mature secreted product showing multiple bands on denaturing protein gel electrophoresis. This is likely due to partial and complete sugar modification on the available N-glycosylation sites (5, 14).

### 3. SUMMARY OF THE INVENTION

The present invention relates to MDA-7 variant proteins which are deficient in or lack glycosylation. It is based, at least in part, on the results of experiments which have demonstrated that such variants are functionally equivalent to wild-type MDA-7 protein. Such proteins may be more easily produced large-scale than the wild-type protein, and may be found to be less immunogenic (thereby facilitating treatment, especially repeated treatments). Accordingly, the present invention provides for such proteins (“Gly(def)MDA-7 proteins”), nucleic acids encoding them (“Gly(def)mda-7 nucleic acids), pharmaceutical compositions comprising such proteins or nucleic acids, and related methods of treatment.

### 4. BRIEF DESCRIPTION OF THE FIGURES

FIG. 1A-D. Comparative growth inhibition, apoptosis induction and MDA-7/IL-24 expression in cells infected with Ad.vec, Ad.mda-7, Ad. SP⁻mda-7 and Ad.SP⁻gly⁻.mda-7. A) Expression vectors and adenoviruses encoding wild type and mutant MDA-7/IL-24. Schematic representation of gene constructs incorporated into the pREP4 expression construct or used to generate recombinant adenoviruses. The full-length mda-7/IL-24 construct encodes a 206-aa protein with a 48-aa signal peptide and three N-glycosylation sites at amino acid position 85, 99 and 126 in the protein. The first three constructs were used to make both pREP4 expression vectors and adenoviruses and the fourth construct was cloned into a pREP4 expression vector. B) Growth inhibition in different tumor cell lines. Cells were infected with 100 pfu/cell of Ad.vec, Ad.mda-7, Ad.SP⁻mda-7 or Ad.SP⁻gly⁻.mda-7 and cell viability was determined by the MTT proliferation assay 5-days after infection. Numbers represent a ratio of specific treatments indicated versus untreated cells. An average of three independent experiments is shown ±S.D. (B, upper panel). Apoptosis induction in cancer cell lines: Cells were treated as described in upper panel and the percentage of the cells displaying hypodiploidy (A₀), a measure of apoptosis, was determined 24 hours later by FACS analysis using the CellQuest software (Becton Dickinson) as described in (15) (B, lower panel). C) MDA-7/IL-24 protein and mda-7/IL-24 mRNA expression in DU-145 cells. Protein lysates were collected from uninfected (control) DU-145 cells and after infection with Ad.vec, Ad.mda-7, Ad.SP⁻mda-7 or Ad.SP⁻gly⁻.mda-7. Samples (50 μg) were run on 12% SDS-PAGE, transferred to a nitrocellulose membrane and stained with rabbit anti-mda-7/IL-24 antibody as described in Materials and Methods. Total RNA was prepared and the expressions of mda-7/IL-24 and GAPDH mRNAs were determined by Northern blotting analysis. D) Protein glycosylation. Protein extracts from Ad.mda-7, Ad.SP⁻mda-7 or Ad.SP⁻gly⁻.mda-7 infected cells were untreated or treated with glycopeptidase F (glyco F) and then evaluated by Western Blot using anti-MDA-7/IL-24 antibody.

FIG. 2A-C. p38^{MAPK} and PKR activation following MDA-7/IL-24 expression in DU-145 cells. A) Activation of the p38^{MAPK} and PKR pathway was determined by Western blotting using total and phospho-specific antibodies 24 hours post-infection with 100 pfu/cell of the different viruses. Additionally, the levels of total and phospho-JNK were determined by Western blotting after viral infection. B) Effect of p38^{MAPK} inhibitor on mda-7/IL-24-induced killing in prostate cancer cell lines. Cells were incubated in the absence or presence of SB203580 (5 μM) after infection with 100 pfu/cell of Ad.vec, Ad.SP⁻gly⁻.mda-7 or Ad. mda-7. Cell viability was determined by MTT assay 6 days after infection. MTT absorbance of untreated control cells was set at 1 to determine relative number of viable cells. Results shown are an average of three independent experiments, bars, ±S.D.C) Apoptotic activity after Ad.vector, Ad.SP⁻gly⁻.mda-7, Ad.SP⁻mda-7, or Ad. mda-7 infection in JAK/STAT deficient cell lines. The cell line indicated at the bottom of each panel was infected with 150 pfu/cell of Ad.vector, Ad.SP⁻gly⁻.mda-7, Ad.SP⁻mda-7, or Ad.mda-7. Cells were analyzed for cell viability by MTT assay 5 days after infection. MTT absorbance of untreated control cells was set at 1 to determine relative number of viable cells. Results shown are an average of three independent experiments.

FIG. 3A-C. Localization of the mutated N-glycosylation deficient MDA-7/IL-24 protein after infection with Ad.SP⁻gly⁻.mda-7.A) MDA-7/IL-24 protein localization was analyzed by indirect immunofluorescence after infection of DU-145 FM-516, C8161, or HO-1 cells with 100 pfu/cell of Ad.SP⁻gly⁻.mda-7 or Ad.vec. Forty eight-hours post infection cells were fixed and MDA-7/IL-24 protein was detected by indirect immunofluorescence using anti-MDA-7/IL-24 antibody. Images of ER were obtained using anti-calreticulin, as described in Materials and methods. Images of the different compartments and MDA-7/IL-24 were merged. B) MDA-7/IL-24 regulates the levels of specific chaperone protein expression. Cells were infected with Ad.vec, Ad.mda-7 or Ad.SP⁻gly⁻.mda-7 and protein changes in BiP/GRP78, calnexin, calreticulin, GRP94, XBP-1, total eIF2α and p-eIF2α were evaluated using Western Blot analyses. C) MDA-7/IL-24 and SP⁻.gly⁻.MDA-7/IL-24 proteins bind to BiP/GRP78. Co-immunoprecipitation of MDA-7/IL-24 protein with BiP/GRP78 protein. DU-145 cells were infected with 100 pfu/cell of Ad.vec, Ad.mda-7 or Ad. SP⁻.gly⁻mda-7 and immunoprecipitation analysis was performed 48 hours later using BiP/GRP78 antibodies.

FIG. 4A-B. ‘Bystander’ suppression of anchorage-independent growth of DU-145 cells following adenovirus infection of P69 cells. Immortal normal P69 cells were seeded in 10-cm plates at a density of 4×10⁶ per plate. The next day, the cells were transfected with various constructs as indicated by lipofectamine, following the manufacturer's protocol (Invitrogen, Lipofectomune 2000 Reagent.) After 24 hours, the transfected cells were trypsinized and reseeded into 6-cm plates at a density of 5×10⁵ cells per plate. The next day, the cells were overlaid with 8-ml of complete DMEM plus 0.3% agar suspended with (A) DU-145 or (B) A549 cells at a density of 1×10⁵ cell per plate. After 6-8 days' incubation with overlay of DMEM plus 0.3% agar every 2-3 days, the colonies >2-mm in size were scored. The data shown are averages of at least 3 plates.

FIG. 5. Proposed pathway of action of full-length and mutated versions of mda-7/IL-24 in inducing apoptosis in cancer cells. Schematic of the role of ER stress and activation of UPR by wild type, signal peptide minus and signal peptide minus plus N-glycosylation mutated MDA-7/IL-24 in inducing apoptosis in DU-145 cells. Under normal conditions, IRE1 and PERK are bound to and inactivate BiP/GRP78. Accumulation of unfolded proteins leads to the release of BiP/GRP78, which is recruited to facilitate folding, and subsequent activation of IRE1 and PERK. As a result, the UPR is initiated, which involves both transcriptional activation of the ER stress-response genes and overall translational repression. Abbreviations: IRE1, inositol-requiring enzyme 1; BiP/GRP78, glucose-regulated protein 78; PERK, PKR-like ER kinase; BAP, BiP-associated protein; ERdj3, stress-inducible endoplasmic reticulum DnaJ homologue; ROS, reactive oxygen species; GADDs, growth arrest and DNA damage inducible genes.

FIG. 6. Amino acid sequence of particular embodiments of Gly(def)MDA-7 (SEQ ID NO:2). Amino acid residues that were Asn at positions 85, 99 and 126 of the original molecule are replaced with Xaa. Each Xaa may be Asn or another naturally occurring or synthetic amino acid, but at least one Xaa must not be Asn. The secretory peptide (residues 1-48), all or a portion of which may be absent in embodiments of Gly(def)MDA-7, is underlined.

### 5. DETAILED DESCRIPTION OF THE INVENTION

For clarity of description and not by way of limitation, the detailed description of the invention is divided into the following sub-sections:
(i) Gly(def)mda-7 nucleic acids;
(ii) Gly(def)MDA-7 proteins;
(iii) expression vectors; and
(iv) uses of the invention.

5.1 GLY(def)MDA-7 Nucleic Acids

The sequence of the wild-type mda-7 gene is as set forth in GenBank Accession No. U16261; Jiang et al., 1995, Oncogene 11:2477-2486, and has a protein coding sequence from nucleotide 275 to nucleotide 895. Gly(def)mda-7 nucleic acids of the invention include nucleic acids comprising nucleotides 419-895 of SEQ ID NO:1, which differs from the wild type sequence in that the three codons represented by nucleotides 527-529, 569-571, and 650-652 may encode any amino acid, but at least one of these three codons does not encode Asn (and preferably at least two do not encode Asn, and more preferably all three do not encode Asn). Said Gly(def)mda-7 nucleic acids may optionally further comprise nucleotides 275-418 (encoding the signal peptide) or a fragment thereof which encodes a peptide that has secretory activity or which does not destroy the antiproliferative activity on HO-1 melanoma cells of the entire encoded protein.

In addition, the present invention provides for Gly(def)mda-7 nucleic acids which are at least 90, at least 95, at least 98, or at least 99 percent homologous (based on standard homology determining software such as BLAST or FASTA) to nucleotides 419-895 of SEQ ID NO:1, provided that, in the sequence of said homolog, at least one of nucleotide triplets corresponding to nucleotides 527-529, 569-571, and 650-652 of SEQ ID NO:1 do not encode Asn, (and preferably at least two do not encode Asn, and more preferably all three do not encode Asn), and such Gly(def)mda-7 nucleic acids encode peptides which exhibit anti-proliferative activity on HO-1 melanoma cells.

In further embodiments of the invention, the present invention provides for Gly(def)mda-7 nucleic acids encoding a polypeptide comprising amino acid residues 1-178 of SEQ ID NO:2, wherein the amino acid residues at positions 37, 51 and 78 may be any amino acid residue, except that at least one of these three residues, preferably at least two of these three residues, and preferably all three residues, is/are not Asn. Said encoded peptide may further comprise amino acids −48-−1 of SEQ ID NO:2 (the signal peptide) or a portion thereof which has secretory activity or which does not destroy the antiproliferative activity of the entire encoded protein on HO-1 melanoma cells.

5.2 GLY(def)MDA-7 Proteins

The wild-type sequence of MDA-7 protein is publicly available as Genbank Accession Number U16261. In non-limiting embodiments, the present invention provides for Gly(def)MDA-7 proteins comprising the amino acid sequence of residues 1-178 of SEQ ID NO:2, where residues 37, 51, and 78 may be any amino acid residue but at least one of these residues, preferably at least two of these residues, and more preferably the three residues is/are not Asn, where said proteins exhibit anti-proliferative activity toward HO-1 melanoma cells. In further non-limiting embodiments, said Gly(def)MDA-7 proteins may further comprise residues −48-−1 of SEQ ID NO:2 (the secretory peptide) or a portion thereof having secretory activity or which does not destroy the antiproliferative activity of the entire protein on HO-1 melanoma cells.

In further non-limiting embodiments, the present invention provides for Gly(def)MDA-7 proteins which comprise a portion which is at least 90 percent, at least 95 percent, or at least 98 percent homologous to residues 1-178 of SEQ ID NO:2, where residues 37, 51, and 78 may be any amino acid residue but at least one of these residues, preferably at least two of these residues, and more preferably the three residues is/are not Asn, where said proteins exhibit anti-proliferative activity toward HO-1 melanoma cells. The portion which is homologous to SEQ ID NO:2 preferably constitutes at least about 90, at least about 95, at least about 98, or 100 percent of said proteins.

5.3 Expression Vectors

In certain preferred embodiments, a Gly(def)mda-7 nucleic acid may be comprised within a larger molecule, such as an expression vector. Thus, to render the Gly(def)mda-7 gene expressible, the nucleic acid may be linked to one of more elements that promote expression. For example, the Gly(def)mda-7 nucleic acid may be operably linked to a suitable promoter element, such as, but not limited to, the cytomegalovirus immediate early (CMV) promoter, the Rous sarcoma virus (RSV) long terminal repeat promoter, the human elongation factor la promoter, the human ubiquitin c promoter, etc. It may be desirable, in certain embodiments of the invention, to use an inducible promoter. Non-limiting examples of inducible promoters include the murine mammary tumor virus promoter (inducible with dexamethasone), commercially-available tetracycline-responsive or ecdysone-responsive promoters, etc. It may also be desirable to utilize a promoter which is selectively active in the cancer cell to be treated, for example the PEG-3 gene promoter (U.S. Pat. No. 6,472,520). Examples of tissue- and cancer cell-specific promoters are well known to those of ordinary skill in the art.

Other elements that may be included in a Gly(def)mda-7-containing nucleic acid and/or vector include transcription start sites, stop sites, polyadenylation sites, ribosomal binding sites, etc.

Suitable expression vectors include virus-based vectors and non-virus based DNA or RNA delivery systems. Examples of appropriate virus-based vectors include, but are not limited to, those derived from retroviruses, for example Moloney murine leukemia-virus based vectors such as LX, LNSX, LNCX or LXSN (Miller and Rosman, 1989, Biotechniques 7:980-989); lentiviruses, for example human immunodeficiency virus (“HIV”), feline leukemia virus (“FIV”) or equine infectious anemia virus (“EIAV”)-based vectors (Case et al., 1999, Proc. Natl. Acad. Sci. U.S.A. 96: 22988-2993; Curran et al., 2000, Molecular Ther. 1:31-38; Olsen, 1998, Gene Ther. 5:1481-1487; U.S. Pat. Nos. 6,255,071 and 6,025,192); adenoviruses (Zhang, 1999, Cancer Gene Ther. 6(2):113-138; Connelly, 1999, Curr. Opin. Mol. Ther. 1(5):565-572; Stratford-Perricaudet, 1990, Human Gene Ther. 1:241-256; Rosenfeld, 1991, Science 252:431-434; Wang et al., 1991, Adv. Exp. Med. Biol. 309:61-66; Jaffe et al., 1992, Nat. Gen. 1:372-378; Quantin et al., 1992, Proc. Natl. Acad. Sci. U.S.A. 89:2581-2584; Rosenfeld et al., 1992, Cell 68:143-155; Mastrangeli et al., 1993, J. Clin. Invest. 91:225-234; Ragot et al., 1993, Nature 361:647-650; Hayaski et al., 1994, J. Biol. Chem. 269:23872-23875; Bett et al., 1994, Proc. Natl. Acad. Sci. U.S.A. 91:8802-8806), for example Ad5/CMV-based E1-deleted vectors (Li et al., 1993, Human Gene Ther. 4:403-409); adeno-associated viruses, for example pSub201-based AAV2-derived vectors (Walsh et al., 1992, Proc. Natl. Acad. Sci. U.S.A. 89:7257-7261); herpes simplex viruses, for example vectors based on HSV-1 (Geller and Freese, 1990, Proc. Natl. Acad. Sci. U.S.A. 87:1149-1153); baculoviruses, for example AcMNPV-based vectors (Boyce and Bucher, 1996, Proc. Natl. Acad. Sci. U.S.A. 93:2348-2352); SV40, for example SVluc (Strayer and Milano, 1996, Gene Ther. 3:581-587); Epstein-Barr viruses, for example EBV-based replicon vectors (Hambor et al., 1988, Proc. Natl. Acad. Sci. U.S.A. 85:4010-4014); alphaviruses, for example Semliki Forest virus- or Sindbis virus-based vectors (Polo et al., 1999, Proc. Natl. Acad. Sci. U.S.A. 96:4598-4603); vaccinia viruses, for example modified vaccinia virus (MVA)-based vectors (Sutter and Moss, 1992, Proc. Natl. Acad. Sci. U.S.A. 89:10847-10851) or any other class of viruses that can efficiently transduce human tumor cells and that can accommodate the nucleic acid sequences required for therapeutic efficacy.

Non-limiting examples of non-virus-based systems which may be used according to the invention include, but are not limited to, so-called naked nucleic acids (Wolff et al., 1990, Science 247:1465-1468), nucleic acids encapsulated in liposomes (Nicolau et al., 1987, Methods in Enzymology 149:157-176), nucleic acid/lipid complexes (Legendre and Szoka, 1992, Pharmaceutical Research 9:1235-1242), nucleic acid/protein complexes (Wu and Wu, 1991, Biother. 3:87-95), bacterial plasmids, yeast plasmids, and bacteriophage.

In specific, non-limiting embodiments of the invention, the expression vector is an E 1-deleted human adenovirus vector of serotype 5, although those of ordinary skill in the art would recognize that many of the different naturally-occurring human Ad serotypes or Ad vectors derived from non-human adenoviruses may substitute for human Ad 5-derived vectors. In a preferred, specific, non-limiting embodiment, a recombinant replication-defective Ad.Gly(def)mda-7 virus for use as a Gly(def)mda-7 vector may be created in two steps as described in Su et al., 1998, Proc. Natl. Acad. Sci. U.S.A. 95:14400-14405. Specifically, a Gly(def)mda-7 nucleic acid may be cloned into a modified Ad expression vector pAd.CMV (Falck-Pedersen et al., 1994, Mol. Pharmacol. 45:684-689). This vector contains, in order, the first 355 by from the left end of the Ad genome, the CMV promoter, DNA encoding splice donor and acceptor sites, the coding region of the mda-7 cDNA, DNA encoding a polyA signal sequence from the β globin gene, and ˜3 kbp of adenovirus sequence extending from within the E1B coding region. This arrangement allows high-level expression of the cloned sequence by the CMV promoter, and appropriate RNA processing. The recombinant virus may be created in vitro in 293 cells (Graham et al., 1977, J. Gen. Virol. 36:59-72) by homologous recombination between a Gly(def)mda-7-containing version of pAd.CMV and plasmid pJM17, which contains the whole of the Ad genome cloned into a modified version of pBR322 (McGrory et al., 1988, Virology 163:614-617). pJM17 gives rise to Ad genomes in vivo, but they are too large to be packaged in mature Ad capsids. This constraint is relieved by recombination with the vector to create a packageable genome (Id.) containing the Gly(def)mda-7 gene. The recombinant virus is replication defective in human cells except 293 cells, which express adenovirus E1A and E1B. Following transfection of the two plasmids, infectious virus may be recovered, and the genomes may be analyzed to confirm the recombinant structure, and then virus may be plaque purified by standard procedures (Volkert and Young, 1983, Virology 125:175-193).

An expression vector comprising Gly(def)mda-7 nucleic acid may be introduced into a host cell for expression, and may be used in therapeutic methods or, using techniques known in the art, to generate a Gly(def)MDA-7 protein for administration to a subject in need of such treatment.

5.4 Uses of the Invention

A Gly(def)mda-7 nucleic acid (optionally comprised in a vector) or a Gly(def)MDA-7 protein may be administered to a host cell or a subject to exert an antiproliferative effect on malignant cells. Said nucleic acid or protein may, for example, be comprised in a pharmaceutical composition together with a suitable carrier (e.g., water, saline, etc.).

As non-limiting examples, said nucleic acid or protein may be administered by intra-tumoral injection, by instillation into the site of a tumor excised by surgery, or may be administered by any other route known in the art, including but not limited to intravenous, intraarterial, intrathecal, hepatic, intra-peritoneal, etc.

Where Gly(def)mda-7 nucleic contained in vector is administered, it may, in non-limiting embodiments, be administered to a population of target cells at a multiplicity of infection (MOI) ranging from 10-100 MOI. In another specific, non-limiting embodiment, the amount of a viral vector administered to a subject may be in the range of 1×10⁹ pfu to 1×10¹² pfu.

Where Gly(def)MDA-7 protein is administered to a subject, in preferred, non-limiting embodiments, said protein may be administered in an amount which achieves a local concentration in the range of between about 18 to 50 ng per microliter. For example, and not by way of limitation, a subject may be administered a range of about 50-100 mg per kilogram (this may, for example, be used per tumor weight for intra-tumoral injection). For a human subject, the dose range may be between 100-2500 mg/treatment or between 1000-2500 mg/day.

Malignant cells, the proliferation of which may be inhibited by Gly(def)mda-7 nucleic acid or protein, include but are not limited to melanoma cells, breast cancer cells, prostate cancer cells, glioblastoma cells, lung cancer cells (adenocarcinoma, small cell, non-small cell, mesothelioma), colon cancer cells, pancreatic cancer cells, cervical cancer cells, fibrosarcoma cancer cells, uterine cancer cells, ovarian cancer cells, hepatic carcinoma cells, gall bladder cancer cells, gastric cancer cells, duodenal cancer cells, renal carcinoma cells, testicular cancer cells, bladder carcinoma cells, leiomyoma cells, sarcoma cells, leukemia cells, and lymphoma cells.

In related embodiments, such methods may be used to treat a subject suffering from a cancer, including but not limited to melanoma, breast cancer, prostate cancer, cervical cancer, uterine cancer, fibrosarcoma, glioblastoma, lung cancer (adenocarcinoma, small cell, non-small cell, mesothelioma), colon cancer, pancreatic cancer, ovarian cancer, hepatic carcinoma, gall bladder cancer, gastric cancer, duodenal cancer, renal carcinoma, testicular cancer, bladder carcinoma, leiomyoma, sarcoma, leukemia, and lymphoma. “Treat” is defined to mean increase duration of survival, increase likelihood of survival for a particular period, increase functionality, increase comfort, decrease tumor mass, or decrease metastasis, each of which effects can be permanent or temporary (for example, a temporary effect may be a temporary increase in functioning or comfort, and may or may not be reinstatable upon repeat administration of the compound), provided that the duration of the affect be clinically acceptable (for example, and not by way of limitation, the duration may be at least one week or at least one month or at least 3 months or at least 6 months).

The foregoing compounds may be administered alone, or may be administered in a regimen that further includes at least one more antineoplastic agent (such as, but not limited to, a chemotherapeutic drug or radiation) which may be administered concurrently or sequentially with molecules according to the invention.

A subject is preferably a human subject.

### 6. EXAMPLE

Material and Methods

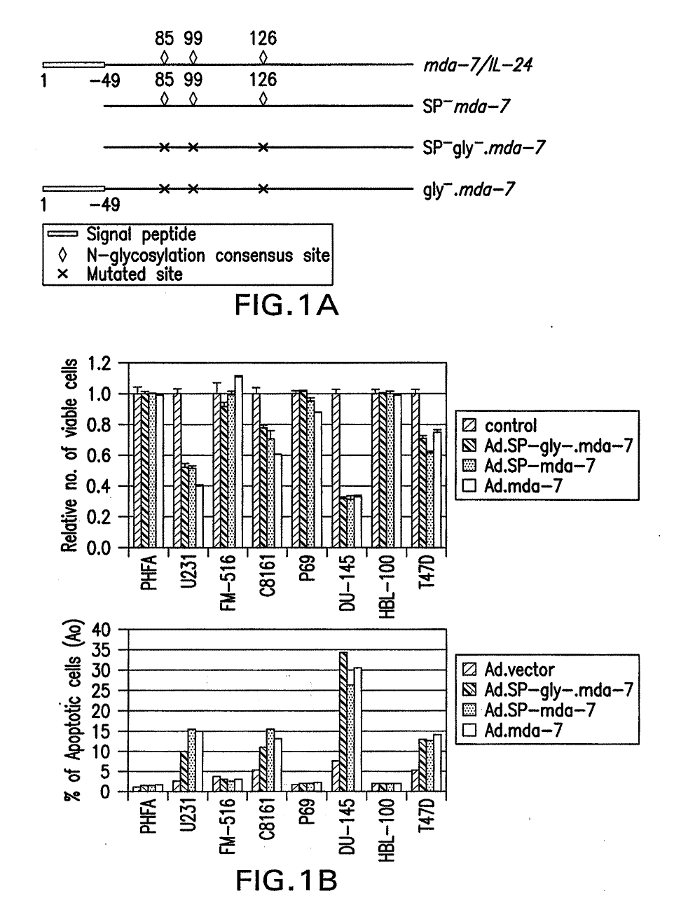

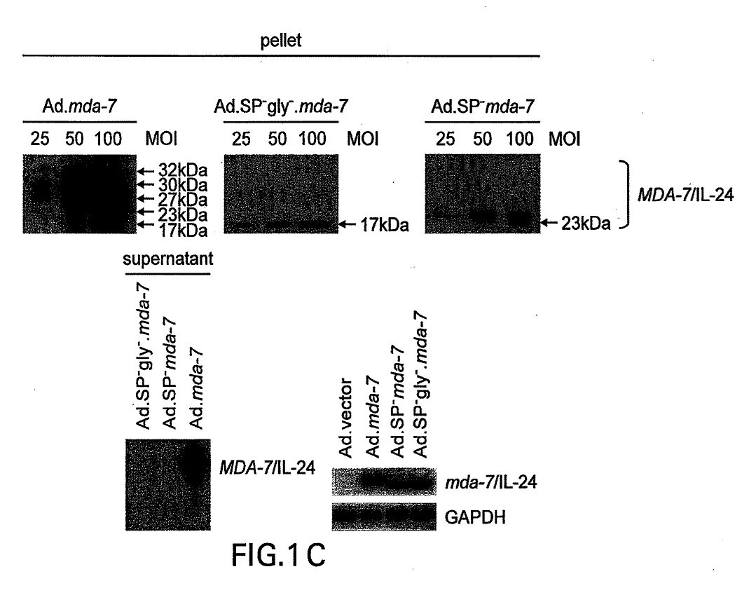

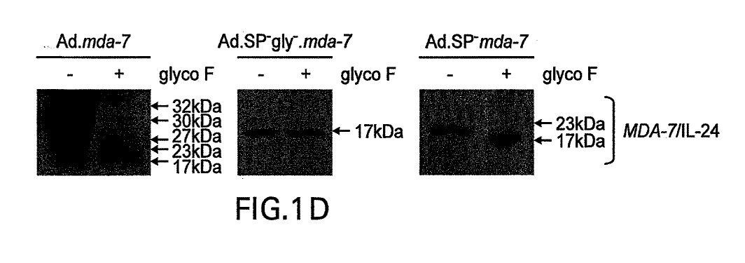

Cell lines, adenoviruses, MTT viability assays, FACS analysis and cell counting: DU-145, U231, T47D, A549, HBL-100, HO-1 and C8161 cells were obtained from the American Type Culture Collection (Rockville, Md.). HO-1 cells were originally obtained from Dr. B. Giovanella (Stehlin Foundation, Houston, Tex.). FM516-SV immortalized normal human melanocytes (referred to as FM-516) were described previously (21). The human fibrosarcoma 2fTGH and its derivative cell lines were provided by Dr. G. Stark (Cleveland Clinic, Cleveland, Ohio) (16) and an immortalized normal human prostate epithelial cell line (P69) (22) was provided by Dr. J. Ware (MCV, Richmond, Va.). Primary human fetal astrocytes (PHFA) were established as previously described and used between passage 3 to 6 (23). Culture and maintenance of cells and construction, propagation and utilization of adenoviruses were as previously described (16). For the present studies, three mda-7/IL-24 cDNAs were engineered in replication incompetent adenoviruses, Ad.mda-7 (encoding a full-length mda-7/IL-24 cDNA), Ad.SP⁻mda-7 (encoding a full-length mda-7/IL-24 cDNA lacking the sequence encoding the signal peptide), and Ad.SP⁻gly⁻.mda-7 (encoding an mda-7/IL-24 cDNA in which all three N-glycosylation sites were mutated and the signal peptide encoding sequence was deleted) (FIG. 1A). The SP⁻gly⁻.mda-7 mutant was generated utilizing a PCR mutagenesis strategy wherein positions 85, 99 and 126 of the MDA-7/IL-24 peptide (SwissProt Accession number Q13007) were mutated from asparagine to glutamine residues so as to prevent N-glycosylation of the mutated protein. The triple mutated MDA-7/IL-24 protein, deleted for the signal peptide was designated SP⁻gly⁻.mda-7. The mutated cDNA was cloned into a non-replicating adenoviral vector, Ad.SP⁻gly⁻.mda-7. MTT assays, FACS analysis and cell counts were performed by standard protocols as previously described (16).

Co-Immunoprecipitation of BiP/GRP78 with MDA-7/IL-24: Cells were infected with Ad.vec. or Ad.mda-7 or transfected with Flag-tagged MDA-7/IL-24 and Myc-tagged BiP/GRP78. After 48 hours, cells were rinsed with ice-cold PBS and lysed in 1 ml of immunoprecipitation buffer containing 25 mM Tris-Cl pH 8.0, 137 mM NaCl, 2.5 mM Kcl, 1% Triton X-100 and protease inhibitor cocktail (Roche, Nutley, N.Y.). The samples were centrifuged ant 4° C. for 10 minutes at 13,000 rpm and the supernatants were incubated with 10 μl of 50% Protein Agarose at 4° C. for 1 hour to eliminate non-specific interactions. Samples were centrifuged and mixed with anti BiP/GRP78 or 9E10 antiMyc monoclonal antibodies (sigma, St. Louis, Mo.; 1:200 dilution) and rotated overnight at 4° C. Immune-complexes were precipitated with 25 μl of 50% protein A agarose for 2 hours. The immunoprecipitates were washed very gently with the IP buffer three times, resuspended in 50 μl of 10 mM Tris-Cl pH 8.0 and 1 mM EDTA and resolved by SDS-PAGE. Western Blot analysis was performed as described (15-17) using the following primary antibodies at 1:1000 dilutions: anti-MDA-7/IL-24, anti-Flag M2, anti-BiP/GRP78 and anti-Myc. Secondary antibodies specific for heavy chain of IgG were used as the light chain of IgG interfered with detection of MDA-7/IL-24 because of its similar size.

Northern blotting analysis: Fifteen-μg of total RNA was denatured, electrophoresed in a 1.2% agarose gel with 3% formaldehyde and transferred onto a nylon membrane. The blots were probed with an α-³²P[dCTP] full-length human mda-7/IL-24 cDNA probe, then stripped and reprobed with an α-³²P[dCTP] human gapdh probe. Following hybridization, the filters were washed and exposed for autoradiography.

Western Blot Analyses: Cell lines were grown on 10-cm plates and protein extracts were prepared with RIPA buffer containing a cocktail of protease inhibitors. Fifty-μg of protein were applied to a 12% SDS-PAGE and transferred to nitrocellulose membranes. The membranes were probed with polyclonal antibodies to MDA-7/IL-24, EF1α, BiP/GRP78, calnexin, calreticulin, GRP94, XBP-1, phospho-JNK, phospho-p38^{MAPK} and total PKR, JNK and p38 antibodies.

“Bystander” Tumor Cell Growth Inhibition Assay: Normal immortal P69 prostate epithelial cells were seeded at 2×10⁵/6-cm dish and transfected with 30 μg of the indicated expression plasmid. The expression plasmids included, pREP4 (control vector plasmid), mda-7 (full-length mda-7/IL-24), SP⁻.mda-7 (mda-7/IL-24 missing the signal peptide), SP⁻.gly⁻.mda-7 (N-glycosylation mutant mda-7/IL-24 gene lacking the signal peptide) or gly⁻.mda-7 (N-glycosylation mutant of mda-7/IL-24 containing the signal peptide) (FIG. 1A). After 24 hours, cells were washed 5× with PBS and overlaid with 6-ml of 0.4% Nobel agar containing 1×10⁵ DU-145 cells. Following 14-days incubation during which overlay cells were referred every 4 days, macroscopic colonies ≧2-mm were scored. Colonies were enumerated from triplicate plates and values expressed as an average±S.D.

Immunofluorescence: Cells were seeded onto chamber slides (Falcon-BD, Calif.) and maintained in DMEM with 10% fetal bOvine calf serum, 24 hours post infection cells were fixed with 2% paraformaldehyde, permeabilized by 0.1% Triton X-100, and then incubated with primary antibodies: rabbit anti-MDA-7/IL-24, GM130 (BD Pharmingen, Calif.), LAMP1/2 (Santa Cruz, Calif.), Calreticulin (BD Pharmingen, Calif.) and Mitrotrack marker (Molecular Probes, Eugene Oreg.). Controls were incubated with only the secondary antibodies under the same experimental conditions. FITC-conjugated donkey anti-mouse IgG or anti-rabbit IgG (Molecular Probes, Eugene Oreg.) were used for visualization on a Zeiss LSM 510 fluorescence microscope.

Results

Comparative growth inhibition and apoptosis induction in cancer cells infected with Ad.SP⁻mda-7, Ad.SP⁻gly⁻.mda-7 and Ad.mda-7. Initial studies determined if infection with an adenovirus expressing a non-glycosylated and non-secreted version of MDA-7/IL-24 protein (Ad.SP⁻gly⁻.mda-7, a non-limiting specific example of a Gly(def)MDA-7 protein) produced growth suppression (loss of viability) and apoptosis in tumor cells in a manner analogous to that observed using a full-length MDA-7/IL-24 protein (Ad.mda-7) or a non-secreted form of the full-length MDA-7/IL-24 protein (Ad.SP⁻mda-7) (FIGS. 1A, B) (16). Parallel experiments were performed with normal primary human fetal astrocytes (PHFA), FM-516, P69, and HBL-100 to substantiate potential differential susceptibility to these viruses. These experiments confirmed that infection with Ad.SP⁻gly⁻.mda-7, Ad.SP⁻mda-7 or Ad.mda-7 induced equivalent growth suppression and decreases in viability in susceptible tumor cell lines (C8161, DU-145, U231 and T47D), without affecting the viability of comparable normal cells (FIG. 1B upper panel).

The mechanism was determined by which Ad.SP⁻gly⁻.mda-7 induced a reduction in growth and decreased survival in cancer cells. For this evaluation, Ad.mda-7 and Ad.SP⁻mda-7 were also included. Infection with all three viruses induced a similar increase in the proportion of tumor cells undergoing apoptosis as reflected by an increase in the percentage of cells with a sub G₀/G₁ (A₀) DNA content (FIG. 1B lower panel). In contrast, no significant change in the percentage of apoptotic cells was found following infection of PHFA, FM-516, P69, and HBL-100 with any of the viruses (FIG. 1B lower panel).

Based on its structure, i.e., absence of a signal peptide, infection with Ad.SP⁻gly⁻.mda-7 (a non-limiting example of a species of a Gly(def)mda-7 nucleic acid, comprised in a viral vector) would be predicted to produce an N-glycosylated mutated MDA-7/IL-24 protein in target cells without promoting secretion. Experiments were performed to confirm this property and to determine the relative levels of mda-7/IL-24 mRNA and MDA-7/IL-24 protein produced following infection with Ad.SP⁻gly⁻mda-7, Ad.SP⁻mda-7 and Ad.mda-7. Northern blot analysis confirmed that comparable levels of MDA-7/IL-24 mRNA were produced following infection with Ad.SP⁻gly⁻.mda-7, Ad.SP⁻mda-7 and Ad.mda-7 (FIG. 1C, lower right panel). Additionally, DU-145 cells were infected with the three viruses and the levels of MDA-7/IL-24 proteins in the supernatants and pellets quantified by Western blotting 24 hours post infection (FIG. 1C). Intracellular protein was observed in extracts of DU-145 cells infected with Ad.SP⁻gly⁻.mda-7, Ad.SP⁻mda-7, or Ad.mda-7. In contrast, secreted MDA-7/IL-24 protein was only detected in the supernatants from Ad.mda-7-infected cell lines at 24, 48 and 72 hours post-infection (see FIG. 1C). Intracellular MDA-7/IL-24 proteins expressed by Ad.SP⁻gly⁻.mda-7 and Ad.SP⁻mda-7 differed from the wild type Ad.mda-7 expressed protein. In Ad.SP⁻gly⁻.mda-7 and Ad.SP⁻mda-7 infected cells a single protein was detected of 17-kDa or 23-kDa, respectively, whereas a series of higher molecular weight species were apparent in Ad.mda-7-infected cells, most likely representing post-translationally processed forms of the MDA-7/IL-24 protein. As predicted, treatment of cell extracts with the enzyme N-glycosidase F, which removes N-linked oligosaccharides, reduced the molecular mass of the 32-, 30-, 27-, and 23-kDa MDA-7/IL-24 proteins to ˜17-kDa (FIG. 1D). Treatment with N-glycosidase F reduced the molecular mass of the MDA-7/IL-24 proteins expressed by Ad.SP⁻mda-7 of 23-kDa to 17-kDa. This 17-kDa species appears to represent the completely deglycosylated form of the MDA-7/IL-24 protein, which does not change following treatment with N-glycosidase F (FIG. 1D).

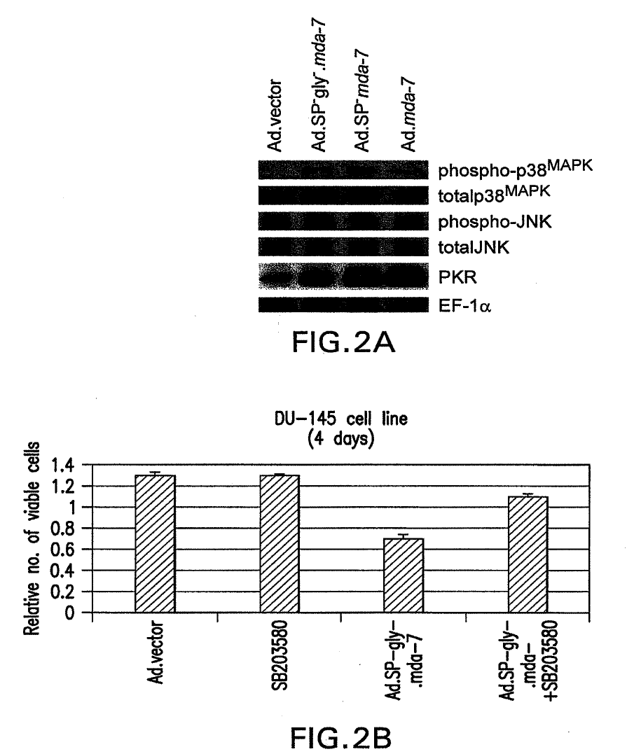

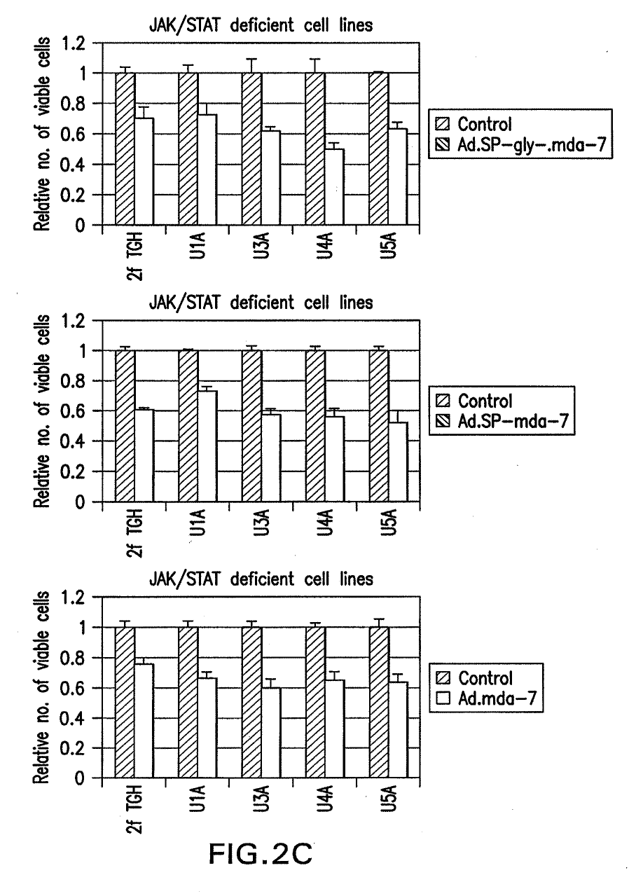

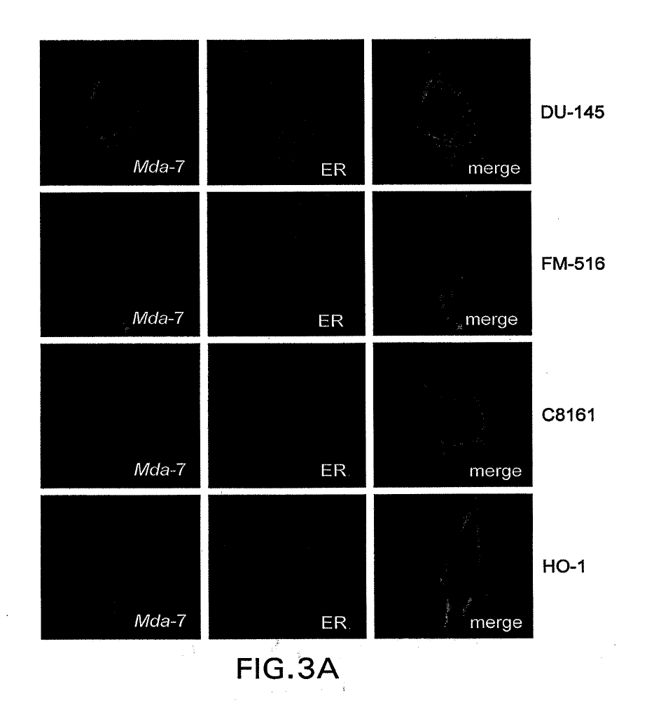

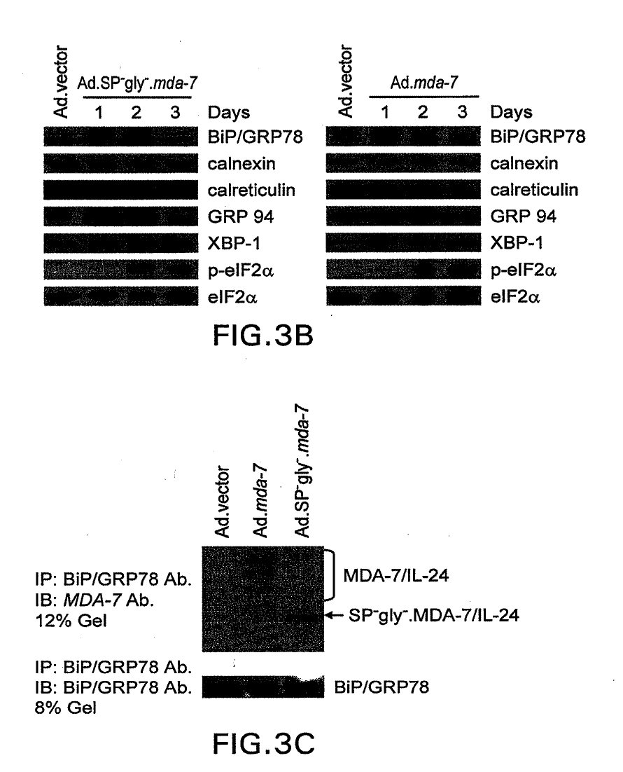

Comparative activation of signal transduction pathways in cells infected with Ad.SP⁻mda-7, Ad.mda-7 and Ad.SP⁻gly⁻.mda-7. Activation of p38^{MAPK} and upregulation of PKR in particular cancer cells upon Ad.mda-7 infection has been shown to be essential in mediating mda-7/IL-24-induced apoptosis in specific tumor cells (24-26). Based on this consideration, it was determined if p38^{MAPK} or PKR activation might also play a role in mda-7/IL-24-induced killing in Ad.SP⁻gly⁻.mda-7 infected cells. DU-145 cells were uninfected or infected with the different viruses and analyzed by SDS-PAGE followed by Western blotting with anti-phospho-JNK, anti-JNK, anti-phospho-p38^{MAPK}, anti-p38^{MAPK}, and anti-PKR antibodies. Treatment with Ad.SP⁻gly⁻.mda-7, Ad.SP⁻mda-7 or Ad.mda-7 promoted p38^{MAPK} phosphorylation in DU-145 cells, whereas it did not affect total p38^{MAPK} (FIG. 2A). Moreover, the specific p38^{MAPK} inhibitor, SB203580, partially blocked the killing effect of Ad.SP⁻gly⁻.mda-7, indicating that p38^{MAPK} is a contributing signaling pathway regulating cell growth and viability (FIG. 2B). Infection of DU-145 cells with the three different adenoviruses expressing mda-7/IL-24 did not alter total levels or phosphorylation of JNK, whereas PKR expression was enhanced. Further studies are being performed to define the relevance of PKR activation by the different viruses expressing variant forms of MDA-7/IL-24. These results document that all three variant mda-7/IL-24-expressing viruses similarly modulate defined and parallel signal transduction pathways in DU-145 cells, namely activation and upregulation of p38^{MAPK} and PKR, respectively. Additionally, as documented previously for Ad.mda-7, the Ad.SP⁻gly⁻.mda-7 virus was capable of decreasing viability in cells deficient in JAK/STAT signaling, further demonstrating the functional equivalence of these viruses (FIG. 3C).

Localization of N-glycosylation mutant MDA-7/IL-24 protein to the ER/Golgi compartments. MDA-7/IL-24 has been reported to localize in the endoplasmic reticulum (ER) subcellular compartment after infection with Ad.mda-7 or Ad.SP⁻mda-7 (16, 17). Experiments were performed to determine if retention of the N-glycosylation sites contribute to this subcellular localization. DU-145, FM-516 (normal SV40-immortalized human melanocyte), C8161 (metastatic melanoma), and HO-1 (metastatic melanoma) cells were infected with Ad.SP⁻gly⁻.mda-7 and subcellular localization of MDA-7/IL-24 protein was determined. The ER was specifically labeled with anti-calreticulin antibody (red color) and MDA-7/IL-24 protein was recognized by green immunofluorescence with a specific anti-MDA-7/IL-24 antibody. Since these signal-peptideless mutants of MDA-7/IL-24 protein do not contain an export signal, they are predicted to remain in the cytosol. However, confocal overlapping imaging results show that MDA-7/IL-24 protein (yellow staining) localizes primarily in the ER (FIG. 3A) indicating that a significant fraction of this protein is able to enter the ER and that proteins derived from wildtype and mutant virus appear to have overlapping patterns of localization within the cell. The possible mechanism of this localization might be (i) the presence of cryptic internalization signals, the identity of which are currently unknown, that become active in the absence of the actual signal peptide and/or (ii) adenovirus infection produces relatively large amounts of protein that even in the absence of a specific targeting sequence possesses the ability to cross membranes and accumulate in the ER/Golgi due to charge and/or tertiary structure.

Regulation of ER chaperone protein levels after Ad.mda-7 or Ad.SP⁻gly⁻.mda-7 infection. Misfolded proteins are retained in the ER and induce an unfolded protein response (UPR) pathway (27). Calnexin and calreticulin are molecular chaperones of the ER that bind to newly synthesized glycoproteins through a lectin site specific for monoglucosylated oligosaccharides, while BiP/GRP78 and GRP94 depend on the presence in proteins of unfolded hydrophobic regions (27). To determine if chaperone proteins were activated after Ad.mda-7 or Ad.SP⁻gly⁻.mda-7 infection, steady-state levels of specific proteins (BiP/GRP78, calnexin, calreticulin, and GRP94) were measured, whose up-regulation frequently correlates with UPR. Additionally, the phosphorylation of eIF2α, a key downstream event of the UPR that mediates inhibition of protein translation, was determined. In addition, the levels of XBP-1 (X-box DNA-binding protein, a UPR-specific b-ZIP transcription factor) (28), whose upregulation and splicing stimulates transcription of specific chaperones, was measured. Since the binding of misfolded proteins to calreticulin depends on the presence of monoglucosylated N-linked glycans, the analysis of this chaperone represents an indirect indicator if glycosylation is essential for mda-7/IL-24-induced apoptosis. Enhanced XBP-1, BiP/GRP78 and GRP94 protein levels were apparent at 1 or 2 days after infection with Ad.mda-7 or Ad.SP⁻gly⁻.mda-7, indicating selective modulation of specific chaperone proteins (FIG. 3B). In addition, Ad.mda-7 and Ad.SP⁻gly⁻.mda-7 induced equivalent phosphorylation of eIF2α.

Previous studies have documented that MDA-7/IL-24 directly interacts with the ER resident chaperone BiP/GRP78 (24). Because full-length MDA-7/IL-24 as well as SP⁻gly⁻MDA-7/IL-24 protein both localize in the ER upon infection with adenovirus, it was investigated whether SP⁻gly⁻MDA-7/IL-24 also interacts with BiP/GRP78, as previously described for full-length MDA-7/IL-24 protein. This was the case since both MDA-7/IL-24 and SP⁻gly⁻MDA-7/IL-24 proteins co-immunoprecipitated with BiP/GRP78 demonstrating a physical interaction between these two molecules (FIG. 3C)

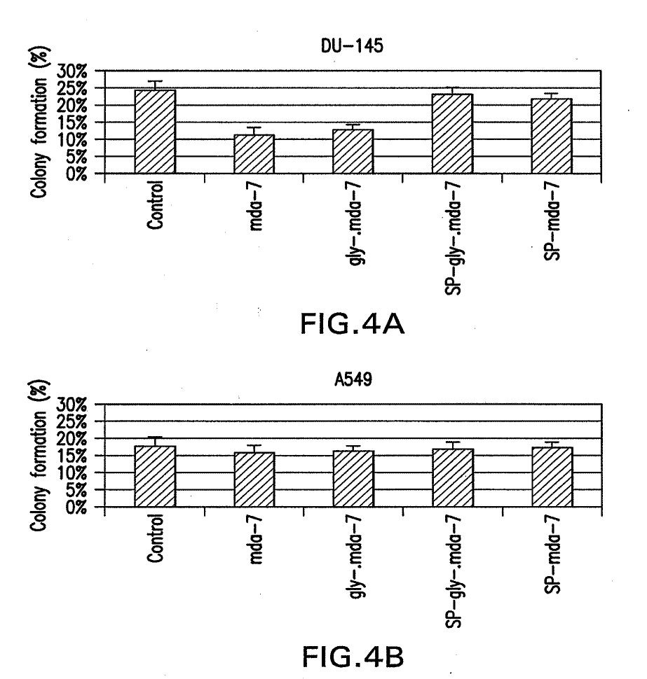

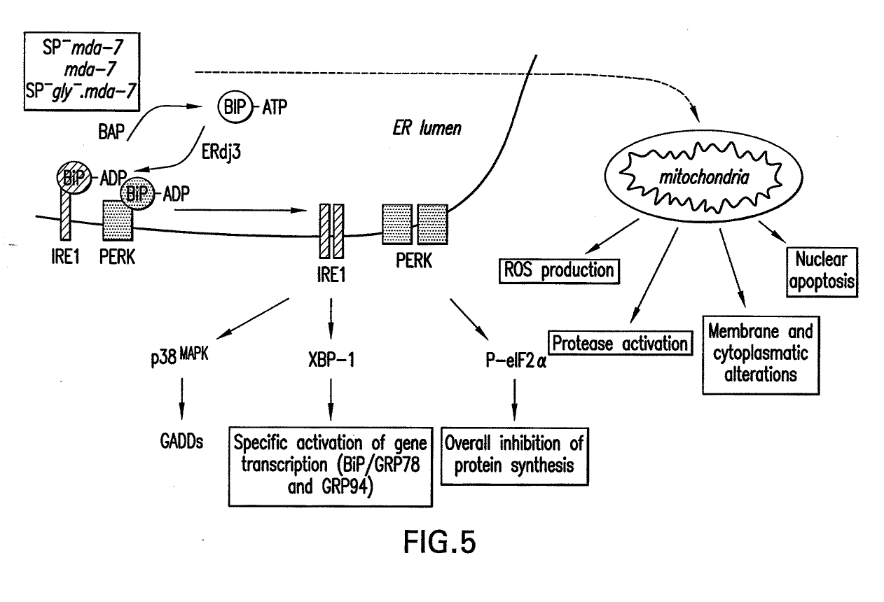

“Bystander” antitumor activity of MDA-7/IL-24 occurs independent of N-glycosylation. A prominent component of mda-7/IL-24 antitumor activity involves its' ability to induce a profound “bystander” antitumor effect. Previous studies have documented that the mechanism of the “antitumor bystander” effect partially differ from the mechanism of tumor cell-specific apoptotic effect mediated by Ad.mda-7 or GST-MDA-7/IL-24 in that “bystander” activity is dependent on the presence of canonical IL-20/IL-22 receptor complexes on target tumor cells, whereas intracellular killing is receptor-independent (29). Based on these considerations it was investigated whether N-glycosylation of MDA-7/IL-24 protein was mandatory for this activity. To determine if the non-glycosylated mutant form of the MDA-7/IL-24 protein could provoke a “bystander” effect on non-expressing cells, a dual normal/tumor cell culture agar overlay diffusion protocol was employed (16, 29). For this assay, normal immortal human prostate epithelial (P69) cells (22), which are resistant to killing by MDA-7/IL-24 while serving as a source of production of this cytokine (16), were transfected with various expression plasmids followed by overlaying with agar containing susceptible DU-145 cells or resistant A549 lung carcinoma cells (FIG. 4). Using this strategy, transfection of P69 cells with full-length mda-7/IL-24 or an expression construct in which the signal peptide of mda-7/IL-24 was incorporated in the gly⁻.mda-7/IL-24 construct (gly⁻.mda-7) (FIG. 1A) resulted in a reduction in both the number and size of DU-145 colonies growing in agar. In contrast, transfection with a control expression vector (not containing an insert) or expression vectors that produce full-length SP⁻MDA-7/IL-24 or SP⁻gly⁻.MDA-7/IL-24 protein that are not secreted did not alter growth of DU-145 cells in the overlay medium. In the case of A549 cells, which do not contain a complete set of IL-20/IL-22 receptors necessary for “bystander” antitumor activity (29), transfection with the various expression constructs had no effect on DU-145 growth in suspension culture (FIG. 4B). These results confirm that N-glycosylation of the MDA-7/IL-24 protein is not required for direct “bystander” antitumor activity.

Discussion

A recent Phase I clinical trial indicates that mda-7/IL-24, administered by adenovirus, is safe and induces significant clinical responses in patients with solid carcinomas and melanomas (6-9). These observations confirm the cancer therapeutic properties of this intriguing cytokine.

In the experiments described above, the role of post-translational modification of MDA-7/IL-24, specifically N-glycosylation, in mediating cancer-selective killing and “bystander” antitumor properties was investigated. In eukaryotic cells, a wide variety of secretory and membrane proteins have one or more N-linked glycans in their outer domains that contribute not only to their conformational maturation but also to their multiple biological functions (18-20). Although it is established that MDA-7/IL-24 has three N-linked glycosylation sites and exists as variably sized protein products, presumably resulting from posttranslational-modifications, the functional significance of these modifications in mediating various biological and biochemical properties of this cytokine have been unknown. Using a series of viruses and expression vectors (FIG. 1A) the experiments described herein demonstrate that N-glycosylation is not required to elicit specific biological and biochemical effects in tumor cells. A mutant MDA-7/IL-24 protein lacking N-glycosylation retains the same localization in the ER/Golgi compartment, stimulates the same signal transduction pathways (namely p38^{MAPK} and PKR) and enhances expression of the same chaperone proteins (namely BiP/GRP78, GRP94 and XBP-1) as does wild-type MDA-7/IL-24 protein. Moreover, when secretion of the mutant N-glycosylated MDA-7/IL-24 protein occurs by adding an mda-7/IL-24 signal peptide to the gly⁻.mda-7 cDNA (gly⁻.mda-7), a similar antitumor “bystander” activity is observed as with the secreted unmodified MDA-7/IL-24 protein (mda-7).

A unique observation for a cytokine is that intracellular MDA-7/IL-24 protein is active in inducing transformed cell-specific apoptosis in a STAT1- and STAT3-independent manner (16). Additionally, multiple studies now indicate that cancer-selective apoptosis induction by MDA-7/IL-24 can involve changes in multiple signal transduction pathways and ER-stress may play a pivotal role in this process (15-17, 24-26). In the experiments described herein, upregulation of both p38^{MAPK} and PKR, but not JNK, is documented in DU-145 cells by both full-length and mutated N-glycosylation deficient MDA-7/IL-24. This observation supports a similar mode of intracellular signal transduction pathway changes leading to apoptosis induction by these two versions of MDA-7/IL-24.

A highly conserved UPR signal transduction pathway is activated by ER-stress caused by misfolded protein accumulation (30-32). The UPR is characterized by the coordinated activation of multiple signal transduction pathways that lead to suppression of the initiation step of protein synthesis, and trigger expression of genes encoding ER chaperones, enzymes and structural components of the ER (31). Prolonged activation of this pathway leads ultimately to apoptosis. The UPR can be triggered by unfolding proteins in the lumen of the ER, resulting in de novo synthesis of ER proteins (such as the “glucose-regulated proteins” BIP/GRP-78 and GRP94) that assist in protein folding. The results herein demonstrate that both N-glycosylated and un-glycosylated MDA-7/IL-24 proteins are equally effective in inducing elevated levels of BiP/GRP78 and GRP94 in DU-145 cells. In contrast, no upregulation of calnexin or calreticulin, which is dependent on the presence of both monoglucosylated N-linked glycans and unfolded regions on nascent glycoproteins, were observed in DU-145 cells infected with viruses expressing wild-type or N-glycosylated mutant MDA-7/IL-24 protein. Additionally, both wild type and N-glycosylated mutant MDA-7/IL-24 enhanced XBP-1 expression and phosphorylation of eIF2α, providing a further link between the activity of MDA-7/IL-24 and induction of UPR and ER stress responses in cancer cells. These results indicate that specific ER stress responses, potentially mediated by upregulation of BiP/GRP78 and GRP94, are elicited by MDA-7/IL-24 and similar changes occur when the protein is un-glycosylated or glycosylated.

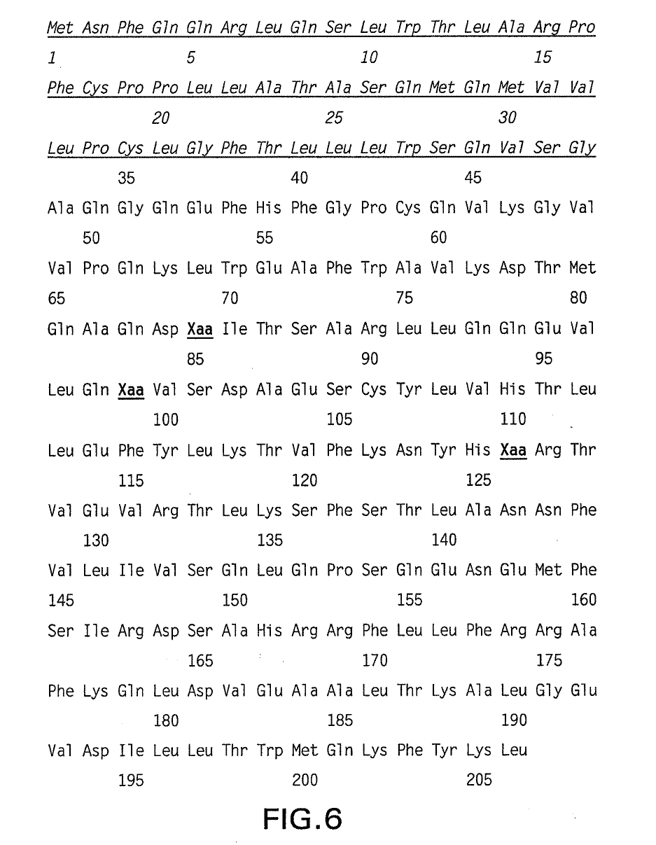

Several additional lines of evidence support the proposal that ER stress, generated by an UPR, is a major factor in eliciting tumor-specific apoptosis by MDA-7/IL-24. Ad.mda-7 infection in cancer cells induces the growth arrest and DNA damage inducible (GADD) gene family, classically associated with the stress response, including the ER-stress pathways (25). Induction of GADD genes and further upstream events such as activation of p38^{MAPK} and PKR are promoted by mda-7/IL-24 in a transformed cell-specific manner and induction of these pathways now appear to occur independent of the glycosylation of MDA-7/IL-24. Additionally, treatment with Ad.mda-7 also specifically activates the p44/42^{MAPK} pathway and produces an up-regulation of the inositol 1,4,5-trisphosphate receptor (IP3R) in H1299 cells (33). IP3R is an intracellular calcium-release channel implicated in apoptosis and localized in the ER. Earlier reports identified putative conserved functional HSP70-like chaperone (BiP/GRP78)-binding sites in both the helix C and F motifs of MDA-7/IL-24 (34). Recent studies demonstrate that mutation(s) of these sites in MDA-7/IL-24 prevent this cytokine from inducing cancer cell-specific apoptosis (24). Additionally, a microarray study indicated that mda-7/IL-24 is able to induce the expression of ER-stress response genes, including BiP/GRP78 (35). Overall, these findings and the results herein suggest a series of events mediated by MDA-7/IL-24 that promote apoptosis, including upregulation of specific signal transduction pathways and gene products involved in the ER stress response (FIG. 6).

As recently emphasized, a significant component of mda-7/IL-24's therapeutic efficacy in vivo involves its ability to induce a potent “bystander” antitumor effect (8, 9, 36). Current studies support the hypothesis that this activity is mediated by a functional set of cell surface receptors consisting of IL-20R1/IL-20R2 and/or IL-22R1/IL-20R2 (29). Similarly, induction of an anti-angiogenic effect by MDA-7/IL-24 also requires these receptors on endothelial cells in the tumor vasculature (37-39). The results herein demonstrate an interesting and potentially important phenomenon that lack of N-glycosylation of the MDA-7/IL-24 protein is not mandatory for antitumor “bystander” activity.

### 7. REFERENCES

1. Jiang H, Fisher P B. Use of a sensitive and efficient subtraction hybridization protocol for the identification of genes differentially regulated during the induction of differentiation in human melanoma cells. Mol Cell Different 1993;1:285-99.
2. Jiang H, Lin J J, Su Z Z, Goldstein N I, Fisher P B. Subtraction hybridization identifies a novel melanoma differentiation associated gene, mda-7, modulated during human melanoma differentiation, growth and progression. Oncogene 1995;11:2477-86.
3. Madireddi M T, Su Z Z, Young C S H, Goldstein N I, Fisher P B. Mda-7, a novel melanoma differentiation associated gene with promise for cancer gene therapy. Adv Exp Med Biol 2000;465:239-61.
4. Fisher P B. Is mda-7/IL-24 a “magic bullet” for cancer? Cancer Res 2005;65:10128-38.
5. Gupta P, Su Z Z, Lebedeva I V, et al. mda-7/IL-24: Multifunctional cancer-specific apoptosis-inducing cytokine. Pharmacol Ther 2006;111:596-628.
6. Lebedeva I V, Sauane M, Gopalkrishnan R V, et al. mda-7/IL-24: exploiting cancer's Achilles' heel. Mol Ther 2005;11:4-18.
7. Fisher P B, Gopalkrishnan R V, Chada S, et al. mda-7/IL-24, a novel cancer selective apoptosis inducing cytokine gene: from the laboratory into the clinic. Cancer Biol Ther 2003;2:S23-37.
8. Cunningham C C, Chada S, Merritt J A, et al. Clinical and local biological effects of an intratumoral injection of mda-7 (IL24; INGN 241) in patients with advanced carcinoma: a phase I study. Mol Ther 2005;11:149-59.
9. Tong A W, Nemunaitis J, Su D, et al. Intratumoral injection of INGN 241, a nonreplicating adenovector expressing the melanoma-differentiation associated gene-7 (mda-7/IL24): biologic outcome in advanced cancer patients. Mol Ther 2005;11:160-72.
10. Dumoutier L, Renauld J C. Viral and cellular interleukin-10 (IL-10)-related cytokines: from structures to functions. Eur Cytokine Netw 2002;13:5-15.
11. Fickenscher H, Hor S, Kupers H, Knappe A, Wittmann S, Sticht H. The interleukin-10 family of cytokines. Trends Immunol 2002;23:89-96.
12. Pestka S, Krause C D, Sarkar D, Walter M R, Shi Y, Fisher P B. Interleukin-10 and related cytokines and receptors. Annu Rev Immunol 2004;22:929-79.
13. Caudell E G, Mumm J B, Poindexter N, et al. The protein product of the tumor suppressor gene, melanoma differentiation-associated gene 7, exhibits immunostimulatory activity and is designated IL-24. J Immunol 2002;168:6041-6.
14. Sauane M, Gopalkrishnan R V, Sarkar D, et al. mda-7/IL-24: novel cancer growth suppressing and apoptosis inducing cytokine. Cytokine Growth Factor Rev 2003;14:35-51.
15. Sauane M, Gopalkrishnan R V, Lebedeva I V, et al. mda-7/IL-24 induces apoptosis of diverse cancer cell lines through JAK/STAT-independent pathways. J Cell Physiol 2003;196:334-45.
16. Sauane M, Lebedeva I V, Su Z Z, et al. Melanoma differentiation associated gene-7/interleukin-24 promotes tumor cell-specific apoptosis through both secretory and nonsecretory pathways. Cancer Res 2004;64:2988-93.
17. Sauane M, Gopalkrishnan R V, Choo H T, et al. Mechanistic aspects of mda-7/IL-24 cancer cell selectivity analysed via a bacterial fusion protein. Oncogene 2004;23:7679-90.
18. Hammond C, Helenius A. Quality control in the secretory pathway. Curr Opin Cell Biol 1995;7:523-9.
19. Helenius, A. How N-linked oligosaccharides affect glycoprotein folding in the endoplasmic reticulum. Mol Biol Cell 1994;5:253-65.
20. Ellgaard L, Molinari M, Helenius A. Setting the standards: quality control in the secretory pathway. Science 1999;286:1882-8.
21. Jiang H, Lin J, Su Z Z, et al. The melanoma differentiation-associated gene mda-6, which encodes the cyclin-dependent kinase inhibitor p21, is differentially expressed during growth, differentiation and progression in human melanoma cells. Oncogene 1995;10:1855-64.
22. Bae V L, Jackson-Cook C K, Brothman A R, Maygarden S J, Ware J L. Tumorigenicity of SV40 T antigen immortalized human prostate epithelial cells: association with decreased epidermal growth factor receptor (EGFR) expression. Int J Cancer 1994;58:721-9.
23. Su Z Z, Kang D C, Chen Y, et al. Identification and cloning of human astrocyte genes displaying elevated expression after infection with HIV-1 or exposure to HIV-1 envelope glycoprotein by rapid subtraction hybridization, RaSH. Oncogene 2002;21:3592-602.
24. Gupta P, Walter M R, Su Z Z, et al. BiP/GRP78 is an intracellular target for MDA-7/IL-24 induction of cancer-specific apoptosis. Cancer Res 2006;66:8182-91.
25. Sarkar D, Su Z Z, Lebedeva I V, et al. mda-7 (IL-24) mediates selective apoptosis in human melanoma cells by inducing the coordinated overexpression of the GADD family of genes by means of p38 MAPK. Proc Natl Acad Sci USA 2002;99:10054-9.
26. Pataer A, Vorburger S A, Barber G N, et al. Adenoviral transfer of the melanoma differentiation-associated gene 7 (mda7) induces apoptosis of lung cancer cells via up-regulation of the double-stranded RNA-dependent protein kinase (PKR). Cancer Res 2002;62:2239-43.
27. Ma Y, Hendershot L M. ER chaperone functions during normal and stress conditions. J Chem Neuroanat 2004;28:51-65.
28. Feldman D E, Chauhan V, Koong A C. The unfolded protein response: a novel component of the hypoxic stress response in tumors. Mol Cancer Res 2005;3:597-605.
29. Su Z Z, Emdad L, Sauane M, et al. Unique aspects of mda-7/IL-24 antitumor bystander activity: establishing a role for secretion of MDA-7/IL-24 protein by normal cells. Oncogene 2005;24:7552-66.
30. Boyce M, Yuan J. Cellular response to endoplasmic reticulum stress: a matter of life or death. Cell Death Differ 2006;13:363-73.
31. Li, J, Lee A S. Stress induction of GRP78/BiP and its role in cancer. Curr Mol Med 2006;6:45-54.
32. Wu J, Kaufman R J. From acute ER stress to physiological roles of the Unfolded Protein Response. Cell Death Differ 2006;13:374-84.
33. Mhashilkar A M, Stewart A L, Sieger K, et al. MDA-7 negatively regulates the beta-catenin and PI3K signaling pathways in breast and lung tumor cells. Mol Ther 2003;8:207-19.
34. Vandenbroeck K, Alloza I, Brehmer D, et al. The conserved helix C region in the superfamily of interferon-gamma/interleukin-10-related cytokines corresponds to a high-affinity binding site for the HSP70 chaperone DnaK. J Biol Chem 2002;25:668-76.
35. Sieger K A, Mhashilkar A M, Stewart A, et al. The tumor suppressor activity of MDA-7/IL-24 is mediated by intracellular protein expression in NSCLC cells. Mol Ther 2004;9:355-67.
36. Sarkar D, Su Z Z, Vozhilla N, Park E S, Gupta P, Fisher P B. Dual cancer-specific targeting strategy cures primary and distant breast carcinomas in nude mice. Proc Natl Acad Sci USA 2005;102:14034-39.
37. Ramesh R, Mhashilkar A M, Tanaka F, et al. Melanoma differentiation-associated gene 7/interleukin (IL)-24 is a novel ligand that regulates angiogenesis via the IL-22 receptor. Cancer Res 2003;63:5105-13.
38. Nishikawa T, Ramesh R, Munshi A, Chada S, Meyn R E. Adenovirus-mediated mda-7 (IL24) gene therapy suppresses angiogenesis and sensitizes NSCLC xenograft tumors to radiation. Mol Ther 2004;9:818-28.
39. Inoue S, Branch C D, Gallick G E, Chada S, Ramesh R. Inhibition of Src kinase activity by Ad-mda7 suppresses vascular endothelial growth factor expression in prostate carcinoma cells. Mol Ther 2005;12:707-15.

Various publications are cited herein, the contents of which are hereby incorporated by reference in their entireties.

We claim:

1. An isolated nucleic acid comprising nucleotides 419-895 of SEQ ID NO:1, which differs from the wild type sequence in that the three codons represented by nucleotides 527-529, 569-571, and 650-652 may encode any amino acid, but at least one of these three codons does not encode asparagine.

2. The nucleic acid of claim 1, wherein at least two of the three codons represented by nucleotides 527-529, 569-571, and 650-652 do not encode asparagine.

3. The nucleic acid of claim 1, wherein none of the three codons represented by nucleotides 527-529, 569-571, and 650-652 encode asparagine.

4. The nucleic acid of claim 1, further comprising nucleotides 275-418 of SEQ ID NO:1 or a portion thereof which encodes a peptide that has secretory activity.

5. An isolated nucleic acid comprising a sequence which is at least about 90 percent homologous to nucleotides 419-895 of SEQ ID NO:1, but which differs from the wild type sequence in that the three codons represented by nucleotides 527-529, 569-571, and 650-652 may encode any amino acid, but at least one of these three codons does not encode asparagine.

6. The nucleic acid of claim 5, wherein at least two of the three codons represented by nucleotides 527-529, 569-571, and 650-652 do not encode asparagine.

7. The nucleic acid of claim 5, wherein none of the three codons represented by nucleotides 527-529, 569-571, and 650-652 encode asparagine.

8. An isolated nucleic acid encoding a polypeptide comprising amino acid residues 49-206 of SEQ ID NO:2, wherein residues 85, 99 and 126 may be any amino acid residue, except that at least one of these three residues is not asparagine.

9. The nucleic acid of claim 8, wherein at least two of residues 85, 99 and 126 are not asparagine.

10. The nucleic acid of claim 8, wherein none of residues 85, 99 and 126 is asparagine.

11. A purified protein comprising the amino acid sequence of residues 49-206 of SEQ ID NO:2, wherein residues 85, 99 and 126 may be any amino acid residue but at least one of these residues is not asparagine.

12. The protein of claim 11, wherein at least two of residues 85, 99 and 126 are not asparagine.

13. The protein of claim 11, wherein none of residues 85, 99 and 126 is asparagine.

14. The protein of claim 11, further comprising residues 1-48 of SEQ ID NO:2 or a portion thereof having secretory activity.

15. A purified protein which is at least 90 percent homologous to residues 49-206 of SEQ ID NO:2, wherein residues 85, 99 and 126 may be any amino acid residue but at least one of these residues is not asparagine.

16. The protein of claim 15, wherein at least two of residues 85, 99 and 126 are not asparagine.

17. The protein of claim 15, wherein none of residues 85, 99 and −126 is asparagine.

18. An expression vector comprising a nucleic acid according to any of claims 1-10.

19. A method of producing an antiproliferative effect on a malignant cell comprising administering, to said malignant cell, an effective amount of a nucleic acid according to any of claims 1-10.

20. A method of producing an antiproliferative effect on a malignant cell comprising administering, to said malignant cell, an effective amount of a protein according to any of claims 11-17.
